(19) United States
(12) Patent Application Publication
(10) Pub. No.: US 2023/0158267 A1
(43) Pub. Date: May 25, 2023

(54) COMPOSITIONS FOR IMPROVING MYALGIC ENCEPHALOMYELITIS/CHRONIC FATIGUE SYNDROME (ME/CFS) AND/OR FIBROMYALGIA (FM) AND/OR INHIBITING WORSENING OF SYMPTOMS

(71) Applicant: MiZ Company Limited, Kamakura-shi (JP)

(72) Inventors: Fumitake SATOH, Kamakura-shi (JP); Yoshiyasu TAKEFUJI, Fujisawa-shi (JP); Takashi YAMAMURA, Tokyo (JP); Bunpei SATO, Kamakura-shi (JP); Shin-ichi HIRANO, Kamakura-shi (JP); Yusuke ICHIAKWA, Kamakura-shi (JP)

(73) Assignee: MiZ Company Limited, Kamakura-shi (JP)

(21) Appl. No.: 17/940,817

(22) Filed: Sep. 8, 2022

(30) Foreign Application Priority Data
Jul. 16, 2021 (JP) 2021-137539
Nov. 24, 2021 (JP) 2021-202927

(51) Int. Cl. A61M 16/10; A61M 15/00; A61M 15/08

(52) U.S. Cl. CPC A61M 16/10; A61M 15/0005; A61M 15/08

(57) ABSTRACT

Described is a composition for ameliorating symptoms of myalgic encephalomyelitis/chronic fatigue syndrome (ME/CFS) and/or fibromyalgia (FM) that has few side effects and can be easily produced. Specifically, described is a composition for ameliorating at least one symptom of fatigue, headache, body pain, impaired thinking, impaired concentration, and/or impaired activity level associated with these symptoms caused by ME/CFS and/or FM in a patient, including a hydrogen gas-containing gas as an active ingredient. Also, described herein is a method for ameliorating symptoms such as fatigue, headache, body pain, impaired thinking, impaired concentration, and/or impaired activity levels associated with these symptoms caused by ME/CFS and/or FM in a patient with ME/CFS and/or FM, including administering the composition to the patient.

### RELATED APPLICATIONS

The present patent document claims the benefit of priority to Japanese Patent Application No. JP2021-137539, filed Jul. 16, 2021 and Japanese Patent Application No. JP2021-202927, filed Nov. 24, 2021, the entire contents of each of which are incorporated herein by reference.

### BACKGROUND OF THE INVENTION

The present invention is intended to be used in human patients with myalgic encephalomyelitis/chronic fatigue syndrome (ME/CFS: Myalgic encephalomyelitis/Chronic fatigue syndrome). ME/CFS and/or fibromyalgia (FM: Fibromyalgia) symptoms include fatigue, headache, body pain, impaired thinking, impaired concentration, and/or decreased activity levels associated with these symptoms.

The invention relates to compositions for improving/alleviating and/or inhibiting the worsening of the above symptoms of ME/CFS and/or FM in human patients.

ME/CFS has a definite time of onset and can be triggered by infections, life-related stress, or severe injuries such as falls and bruises. It is triggered by severe general malaise, followed by long-term fatigue, extreme post-exertional malaise, low-grade fever, headache, myalgia, weakness, cognitive dysfunction, sleep without restoration, and dizziness.

FM is primarily characterized by generalized pain of unknown cause. The pain extends to tendon adhesions, muscles, and joints, and the disease is characterized by severe pain that spreads from the trunk and extremities to the entire body.

The symptoms of both, ME/CFS and FM are beyond description, and are characterized by the inability of patients with ME/CFS and/or FM to engage in healthy social activities. However, the pathological mechanisms of ME/CFS) and/or FM are unknown and there is no cure. In the United States, the cause of ME/CFS was thought to be due to a viral infection. However, in recent years, the ME/CFS has been recognized as a sequela of the new coronavirus infection (COVID-19) that originated in Wuhan, China. ME/CFS is reported to affect approximately 300,000 people in Japan and 800,000 to 3.5 million people in the United States. The number of patients in the U.S. is said to range from 800,000 to 3.5 million (“Pathomechanisms of Chronic Fatigue Syndrome and Its Treatment,” Yasuyoshi Watanabe and Hirohiko Kuragaki, Neurotherapy, Vol. 33, No. 1, 2016).

FM, along with ME/CFS, is a disease with no clear cause. However, both diseases share common symptoms such as indescribable pain, fatigue that is qualitatively different from overwork, impaired thinking, and abnormal sensory sensitivity, as described above. Some physicians treat them as related diseases and consider them to be the same illness (Journal of Insurance and Medical Sociology, Vol. 27, No. 2, 2017, “Diagnostic Paradox: Myalgic Brain Effects and Limitations of Diagnosis in People with Myalgic Encephalomyelitis/Chronic Fatigue Syndrome and Linear Myalgia,” Hisasaki Nishioka; “Fibromyalgia to Tatakau” (Fighting Fibromyalgia), Wholeness Study Group, 2004, p. 108.).

### SUMMARY OF THE INVENTION

The purpose of this invention is to provide new compositions for ameliorating symptoms and/or inhibiting worsening of symptoms of myalgic encephalomyelitis/chronic fatigue syndrome (ME/CFS) and/or fibromyalgia (FM) that can be easily produced with few side effects.

Recently, it has been proposed that molecular hydrogen may inhibit chronic inflammation by scavenging hydroxyl radicals generated inside mitochondria of cells, and may be effective against many diseases caused by chronic inflammation. However, it has not been reported whether the symptoms of ME/CFS and/or FM are ameliorated and/or the worsening of symptoms is inhibited when human ME/CFS and/or FM patients inhale or drink gas containing hydrogen gas.

After diligent research, the inventors have surprisingly found that hydrogen gas-containing gases ameliorate certain symptoms of ME/CFS and/or FM and/or inhibit the worsening of symptoms in human patients.

Accordingly, the present invention encompasses the following features:

(1) Composition containing hydrogen gas-containing gas as an active ingredient for ameliorating symptoms caused by ME/CFS and/or FM and/or inhibiting worsening of symptoms in patients with ME/CFS and/or FM.

(2) The composition according to (1), wherein the ME/CFS causing symptoms include at least one of fatigue, tiredness, shortness of breath with exertion, palpitations, muscle weakness, slight fever, muscle pain, joint pain, sore throat, headache, physical pain, generalized pain, weakness, cognitive dysfunction, memory impairment, difficulty with language retrieval, gastrointestinal disturbances, impaired concentration, flu-like symptoms, dizziness on standing, weakness, irregular body temperature, cold hands and feet, intolerance to heat and/or cold, perspiration, facial flushing, lymph node tenderness, significant weight fluctuations, tinnitus, confusion, sleep disturbances, dizziness when standing up, dizziness, hypersensitivity to light, noise, or odors, new acquired sensitivity to food, drugs, or chemicals, weakness, fatigue after exertion, bradycardia, decreased blood pressure, nausea, brain fog and/or decreased activity level associated with these symptoms.

(3) The composition according to (1), wherein the FM causing symptoms include at least one of slight fever of 38° C. or less, fatigue, tiredness weakness, stiffness of the hands and fingers, swollen fingers, joint pain, Raynaud's phenomenon, night sweats, irritable bowel syndrome, palpitations, dryness symptoms, dyspnea, dysphagia, interstitial cystitis-like symptoms, menstrual irregularities, dysmenorrhea, weight fluctuation, photosensitivity, intolerance to cold and heat, temporomandibular joint disorder, low blood pressure, various allergic symptoms, mitral valve deviation, itching, numbness in the extremities, hand tremors, dizziness, tinnitus, hearing loss, vision impairment, depressive symptoms, anxiety, agitation, sleep disturbances (hypersomnia, insomnia), decreased concentration, decreased attention span, amnesia, discomfort upon awakening, muscle weakness, decreased ability to think, concentrate, and remember, hypersensitivity to sound, brain fog, and/or decreased activity level associated with these symptoms.

(4) The composition according to any one of (1) or (2), wherein the cause of the ME/CFS is a viral infection.

(5) The composition according to any one of (1) to (4), wherein the hydrogen concentration of the hydrogen gas-containing gas is greater than zero (0) and less than 18.5 volume percent.

(6) The composition according to any one of (1) to (5), wherein the composition is administered to said patient by inhalation.

(7) A method for making a composition according to any one of the preceding (1) to (6), wherein the composition is made using a hydrogen gas generator.

(8) A method for ameliorating a symptom of myalgic encephalomyelitis/chronic fatigue syndrome (ME/CFS) and/or fibromyalgia (FM) and/or inhibiting worsening of a symptom in a patient with ME/CFS and/or FM, comprising: administering to the patient a composition comprising hydrogen gas-containing gas, the hydrogen gas being the active ingredient.

### Effects of Invention

The described compositions and methods can significantly improve/ameliorate and/or inhibit the worsening of symptoms such as fatigue, headache, body pain, poor thinking, poor concentration, and/or reduced activity levels associated with these symptoms, at least due to ME/CFS and/or FM, by absorption, inhalation, etc., of hydrogen gas.

### DETAILED DESCRIPTION OF THE PREFERRED EMBODIMENTS

The present invention will be described in more detail below.

### 1. Myalgic Encephalomyelitis/Chronic Fatigue Syndrome (ME/CFS) and/or Fibromyalgia (FM)

ME/CFS is classified as an intractable neurologic disease, the symptoms of which include fatigue, malaise, shortness of breath with exertion, palpitations, muscle weakness, low-grade fever, myalgia, arthralgia, sore throat, headache, body aches, weakness, cognitive dysfunction, memory impairment, difficulty retrieving language, gastrointestinal disturbances, poor concentration, influenza-like symptoms, dizziness on standing, weakness, irregular body temperature, cold hands and feet, intolerance to heat and/or cold, sweating, facial flushing, tender lymph nodes, significant weight fluctuations, tinnitus, confusion, sleep disturbances, dizziness, sensitivity to light, noise or odor, new sensitivities to food, drugs or chemicals, and/or or decreased activity levels associated with these symptoms (“Pathomechanisms of Chronic Fatigue Syndrome and Its Treatment,” Yasuyoshi Watanabe and Hirohiko Kuragaki, Neurotherapy, Vol. 33, No. 1, 2016).

In the United Kingdom and Canada, many patients with chronic fatigue syndrome (CFS)-like conditions present with muscle pain and a variety of clinical symptoms that may be due to cranial nerve abnormalities myalgic encephalomyelitis/chronic encephalomyelitis/chronic myalgic encephalomyelitis (ME).

According to the internationally agreed-upon diagnostic criteria used to diagnose ME/CFS the main symptoms reported by patients with myalgic encephalomyelitis/chronic fatigue syndrome are: a) extreme exhaustion of the neuroimmune system after exertion; b) nervous system dysfunction; c) immune, gastrointestinal, and genitourinary disorders; and d) dysfunction of energy production/transport over a prolonged period of time (generally 6 months or more).

The ME/CFS symptoms that can be improved/ameliorated and/or prevented from worsening by the present invention are those listed in the diagnostic guidelines of the Japan Fatigue Society and/or the Canadian Diagnostic Guidelines, such as fatigue, shortness of breath with exertion, palpitations, muscle weakness, slight fever, muscle pain, joint pain, sore throat, headache, physical pain, generalized pain, weakness, cognitive dysfunction, memory impairment, difficulty with language retrieval, gastrointestinal disturbances, difficulty concentrating, flu-like symptoms, dizziness on standing, weakness, irregular body temperature, cold hands and feet intolerance to heat and/or cold, perspiration, facial flushing, lymph node tenderness, significant weight, fluctuations, tinnitus, confusion, sleep disturbances, lightheadedness, dizziness, hypersensitivity to light, noise, or odors, new acquired sensitivity to food, drugs, or chemicals, weakness, fatigue after exertion, bradycardia, decreased blood pressure, nausea, brain fog and/or decreased activity level associated with these symptoms.

Exemplary causes of ME/CFS may be viral infections, such as coronaviruses, including the novel coronavirus that caused COVID-19 and pathogenic viruses such as influenza virus, norovirus, Ebola virus, Marburg virus, hantavirus, AIDS virus, papillomavirus, and herpes virus.

Fibromyalgia is a disease characterized by generalized pain of unknown cause. Fibromyalgia is a disease characterized by severe pain that extends from the trunk and extremities to the entire body, including tendonitis, muscles, and joints. Other symptoms of FM may include low-grade fever of 38° C. or less, fatigue, malaise, weakness, stiffness of the fingers, swelling of the fingers, joint pain, Raynaud's phenomenon, night sweats, irritable bowel syndrome, palpitations, dryness, dyspnea, dysphagia, interstitial cystitis-like symptoms, irregular periods, dysmenorrhea weight fluctuation, photosensitivity, cold intolerance, temporomandibular joint disorder, hypotension, various allergic symptoms, mitral valve deviation, itching, numbness in extremities, finger tremor, dizziness, tinnitus, hearing loss, vision impairment, depression, anxiety, agitation, sleep disorder (hypersomnia, insomnia), poor concentration, poor attention span, forgetfulness, discomfort upon waking, muscle weakness, poor thinking, concentration, and memory, hypersensitivity to sound, brain fog, and/or decreased activity levels associated with these symptoms.

FM, along with ME/CFS, is a disease with no known cause. However, both diseases share common symptoms such as indescribable pain, fatigue that is qualitatively different from overwork, impaired thinking, and abnormal sensory sensitivity, and some doctors consider them to be the same disease. FM and ME/CFS are considered closely related diseases (Journal of Insurance and Medical Sociology, Vol. 27, No. 2, 2017, “Diagnostic Paradox: Myalgic Brain Effects and Limitations of Diagnosis in People with Myalgic Encephalomyelitis/Chronic Fatigue Syndrome and Linear Myalgia,” Hisasaki Nishioka, “Fibromyalgia to Tatakau” (Fighting Fibromyalgia), Wholeness Study Group, 2004, p. 108).

### 2. Compositions for Improving/Ameliorating Symptoms and/or Inhibiting Worsening of Symptoms of Myalgic Encephalomyelitis/Chronic Fatigue Syndrome (ME/CFS) and/or Fibromyalgia (FM), Including Gases Containing Hydrogen Gas

Certain embodiments relate to a hydrogen gas-containing gas as an active ingredient for use in patients with myalgic encephalomyelitis/chronic fatigue syndrome (ME/CFS) and/or fibromyalgia (FM), wherein ME/CFS and/or FM are characterized by at least fatigue, malaise, shortness of breath with exertion, palpitations, muscle weakness, slight fever, myalgia, arthralgia, sore throat, headache, body pain, weakness, cognitive dysfunction, memory impairment, language retrieval difficulties, gastrointestinal disturbances, poor concentration, flu-like symptoms, dizziness on standing, weakness, irregular body temperature, cold hands and feet, intolerance to heat and/or cold, sweating, facial flushing, tender lymph nodes, significant weight changes, tinnitus, confusion, sleep disturbances, orthostatic. The compositions are provided to ameliorate and/or inhibit the worsening of symptoms, including dizziness, sensitivity to light, noise or odor, new sensitivities to food, drugs or chemicals, and/or decreased activity levels associated with these symptoms.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas.

The concentration of hydrogen gas in a gas containing molecular hydrogen is higher than zero (0) and not higher than 18.5% by volume, for example, 0.5% to 18.5% by volume, preferably 1% to 10% by volume, for example, 2% to 8% by volume, 2% to 9% by volume, 2% to 10% by volume, 3% to 6% by volume, 3% to 7% by volume, 3% to 8% by volume, 3% to 9% by volume, 3% to 10% by volume, 4% to 5% by volume, 4% to 6% by volume, 4% to 7% by volume, 4% to 8% by volume, 4% to 9% by volume, 4% to 10% by volume, 5% to 8% by volume, 5% to 9% by volume, 5% to 10% by volume, 6% to 7% by volume, 6% to 8% by volume, 6% to 9% by volume, 6% to 10% by volume, and the like. In the present invention, higher hydrogen gas concentration (but equal to or less than the explosion limit) or larger daily hydrogen doses tend to be associated with greater effects of maintaining or improving ME/CFS and/or FM.

In the present invention, a hydrogen gas supply apparatus which generates and emits hydrogen in the safety concentration above should be used.

Since hydrogen is a flammable and explosive gas, it is preferable to include it in the described composition and administer it to ME/CFS and/or FM patients under safe conditions that would allow for improving/ameliorating ME/CFS and/or FM symptoms and/or inhibiting worsening of symptoms.

In an embodiment, where the gas other than hydrogen gas is air, the concentration of air is, for example, in the range of 81.5 to 99.5% by volume.

In another embodiment, where the gas other than hydrogen gas is a gas containing oxygen gas, the concentration of oxygen gas is in the range of 21-99.5 volume percent, for example.

In certain embodiments, nitrogen gas can be included as the other main gas.

In further embodiments, gases such as carbon dioxide, which is a gas contained in air, may be included in amounts about the same as present in air.

In the present invention, hydrogen-dissolved liquids can be administered or ingested by ME/CFS patients or FM patients in conjunction with the administration of hydrogen gas-containing gases, if necessary.

When co-administered with a hydrogen-dissolved liquid, the composition may be administered before, simultaneously with, or after administration of the hydrogen-dissolved liquid.

The hydrogen-dissolved liquid is specifically an aqueous liquid in which hydrogen gas is dissolved, wherein the aqueous liquid is, non-limiting, for example, water (e.g., sterile water, purified water), saline solution, buffer solution (e.g., pH 4 to 7.4 buffer solution), ethanol-containing water (e.g., ethanol content of 0.1 to 2% by volume), intravenous solution, infusion solution, injection solution, beverage, etc. The hydrogen concentration of the hydrogen-dissolved liquid is, for example, 1-10 ppm, e.g., 2-8 ppm, 3-7 ppm, 3 to 7 ppm, 3 to 6 ppm, 4 to 6 ppm, 4 to 5 ppm, 5 to 5 ppm, 5 to 10 ppm 4 to 5 ppm, 5 to 10 5 to 10 ppm, 5 to 8 ppm, 6 to 8 ppm, 6 to 7 ppm, 6 to 7 ppm, etc., 8 ppm, 6-7 ppm, etc., more preferably 5 to 8 ppm, e.g., 6 to 8 ppm, 6 to 7 ppm, etc., 5 to 8 ppm, e.g., 6 to 8 ppm, 6 to 7 ppm, etc. In the present invention, the higher the concentration of hydrogen gas below the explosive limit, the more likely it is to be more effective in improving/ameliorating symptoms and/or inhibiting worsening of symptoms ME/CFS and/or FM.

In certain embodiments, pharmaceuticals to treat ME/CFS or FM may be added to the hydrogen solution liquid. Alternatively, said pharmaceuticals may be administered separately from the administration of the hydrogen-dissolved liquid or hydrogen gas-containing gas.

The hydrogen gas-containing gas or hydrogen-dissolved liquid may be blended to a predetermined hydrogen gas concentration and then filled into pressure-resistant containers (e.g., stainless steel cylinders, aluminum cans, preferably laminated with aluminum film on the inside, pressure-resistant plastic bottles (e.g., pressure-resistant PET bottles) and plastic bags, aluminum bags, etc.). Aluminum has the property of being impermeable to molecular hydrogen. Alternatively, the hydrogen gas-containing gas or hydrogen-dissolved liquid can, at the time of administration, be supplied to a hydrogen gas generator, hydrogen water generator, or hydrogen gas adding device, such as a known or commercially available hydrogen gas supply device (for generating hydrogen gas-containing gas), hydrogen adding apparatus (for generating hydrogen water), non-destructive hydrogen containing device (such as an intravenous solution It may be made in-situ using a device such as a non-destructive hydrogen gas adding apparatus (a device for non-destructively adding hydrogen gas to the inside of a biological application solution bag, such as intravenous solution).

The hydrogen gas supply device allows the hydrogen gas generated by the reaction of a hydrogen generating agent (e.g., aluminum metal, magnesium hydride, etc.) and water to be mixed with a dilution gas (e.g., air, oxygen, etc.) in a predetermined ratio (JP5228142(B), etc.). Alternatively, hydrogen gas generated by electrolysis of water is mixed with oxygen, air, or other gases for dilution (JP5502973(B), JP5900688(B), etc.). By this method, a hydrogen gas-containing gas with a hydrogen concentration in the range of 0.5 to 18.5 volume percent can be prepared.

A hydrogen adding apparatus is a device that uses a hydrogen generating agent and a pH adjuster to generate hydrogen and dissolve it in a biological application solution, such as water (JP4756102(B), JP4652479(B), JP4950352(B), JP6159462(B), JP6170605(B), JP2017-104842(A), etc.). The combination of hydrogen generating agent and pH adjuster is, for example, metallic magnesium and strong acidic ion exchange resin or organic acid (e.g., malic acid, citric acid, etc.), metallic aluminum powder and calcium hydroxide powder, etc. This allows the preparation of a hydrogen-dissolved liquid with a dissolved hydrogen concentration of 1 to 10 ppm (e.g., product name “Seven Water” (Quassia), etc.).

A nondestructive hydrogen-containing device or apparatus is a commercially available bio-applicable solution, such as intravenous solution (e.g., sealed in a hydrogen-permeable plastic bag such as a polyethylene bag). The device or apparatus adds molecular hydrogen to the solution from outside the package, for example, is commercially available from MiZ Corporation (http://www.e-.miz.co.jp/technology), (http://www.e-miz.co.jp/technology.html). This device can aseptically dissolve hydrogen in the bio-applied solution by immersing a bag containing the bio-applied solution in saturated hydrogen water until hydrogen permeates into the bag and the concentration equilibrium is reached. The apparatus may, for example, comprise an electrolyzer and a water tank, and water in the water tank can circulate between the electrolyzer and the water tank to produce hydrogen by electrolysis. Alternatively, a simple disposable apparatus can be used for the same purpose (JP2016-112562(A), etc.). This apparatus contains a plastic bag (hydrogen permeable bag, e.g., polyethylene bag) containing a bio-applicable solution and a hydrogen generating agent (e.g., metallic calcium, metallic magnesium/cation exchange resin, etc.) in an aluminum bag, with the hydrogen generating agent wrapped in a nonwoven fabric, e.g., water vapor permeable nonwoven fabric. The hydrogen generating agent is wrapped in a nonwoven fabric (e.g., water vapor permeable nonwoven fabric). By wetting the hydrogen generating agent wrapped in the non-woven fabric with a small amount of water, such as water vapor, the generated hydrogen permeates the plastic bag and is non-destructively and aseptically dissolved in the bio-applied solution.

The hydrogen gas-containing gas or hydrogen-saturated bio-applied solution (e.g., sterile water, saline solution, intravenous solution, etc.) prepared using the device or apparatus described above can be administered orally or parenterally to ME/CFS and/or FM patients.

Another form of the compositions of the invention includes dosage forms (e.g., tablets, capsules, etc.) containing hydrogen generating agents that are prepared for oral administration (or ingestion) to ME/CFS and/or FM patients and that allow hydrogen generation in the digestive tract. The hydrogen generating agent should preferably comprise ingredients that are approved, for example, as food or food additives.

### 3. Improvement of Symptoms and/or Preventing Worsening of Symptoms of Myalgic Encephalomyelitis/Chronic Fatigue Syndrome (ME/CFS) and/or Fibromyalgia (FM)

In another embodiment described herein is a method that includes administering to a patient with ME/CFS and/or FM the described composition, wherein the patient has at least one of fatigue, malaise, shortness of breath with exertion, palpitations, muscle weakness, low fever, myalgia, arthralgia, sore throat, headache, body aches, weakness, cognitive dysfunction memory impairment, language retrieval difficulties, gastrointestinal disturbances, poor concentration, flu-like symptoms, dizziness on standing, weakness, irregular body temperature, cold hands and feet, intolerance to heat and/or cold, sweating, facial flushing, tender lymph nodes, significant weight fluctuations, tinnitus, confusion, sleep disturbances, lightheadedness, sensitivity to light, noise, or hypersensitivity to light, noise or odors, acquisition of new sensitivities to food, drugs or chemicals, and/or methods to ameliorate and/or inhibit the worsening of symptoms, including decreased activity levels associated with these symptoms.

The described compositions allow for improvement of the patient's quality of life (QOL).

In certain embodiments, the described composition, when hydrogen gas is used as an active ingredient, may be administered to a patient with ME/CFS and/or FM, preferably, for example, via pulmonary administration by absorption, inhalation, or the like. When a gas is inhaled, it can be inhaled through the mouth or nose, through a nasal cannula or a mask-type device that covers the mouth and nose and is delivered to the lungs for systemic delivery via the blood.

When hydrogen-dissolved liquids are administered to patients, oral, intravenous, or intra-arterial (including intravenous infusion) administration is preferred. For orally administered hydrogen-dissolved liquid, the liquid may be administered to a human ME/CFS and/or FM patient either cooled, preferably stored at a low temperature, or stored at room temperature. Hydrogen is dissolved in water at a concentration of about 1.6 ppm (1.6 mg/L) in water, and it is known that the difference in solubility with temperature is relatively small. Alternatively, the hydrogen-dissolved liquid may be administered to ME/CFS and/or FM patients by parenteral routes of administration, such as intravenous or intra-arterial administration, when it is in the form of an infusion or injection solution containing hydrogen gas prepared, for example, with the non-destructive hydrogen-containing device described above.

The gas containing hydrogen gas at the above hydrogen concentration or the hydrogen-dissolved liquid at the above dissolved hydrogen concentration may be administered to the patient body of ME/CFS or FM once or more times per day (e.g., two to three times) for a period of one week to three months or longer, such as one week to six months or longer. When hydrogen gas-containing gases are administered, they may be administered over a period of, for example, 10 minutes to 2 hours or more per dose, preferably 20 minutes to 40 minutes or more, preferably over a period of 30 minutes to 2 hours. When hydrogen gas-containing gases are administered via inhalation, they can be administered to ME/CFS patients under atmospheric pressure. Or, the gas can be administered to ME/CFS patients at high pressure in the range from standard atmospheric pressure (Approx. 1.013 atmospheres) to 7.0 atmospheres or less. In addition, the gas can be administered to ME/CFS patients under hyperbaric conditions in the range of 1.02 to 7.0 atmospheres, preferably 1.02 to 5.0 atmospheres, more preferably 1.02 to 4.0 atomospheres, even more preferably 1.02 to 1.35 atm.

The above hyperbaric environment can be created by the use of a hyperbaric enclosure (e.g., a capsule-like enclosure) designed to have sufficient strength to allow the hydrogen gas-containing gas described above (e.g., hydrogen-containing oxygen or air) to be pressurized inside to create an internal hyperbaric environment above standard atmospheric pressure and below 7.0 atm. The shape of the hyperbaric enclosure can be made by the use of a capsule-shaped enclosure. The shape of the hyperbaric enclosure should be rounded with no corners overall, as it is pressure-resistant. The material of the hyperbaric enclosure should be lightweight and strong, for example, reinforced plastic, carbon fiber composite, titanium alloy, aluminum alloy, etc. Patients may receive the composition in the above hyperbaric enclosure containing oxygen gas or air and hydrogen gas to improve and/or inhibit the worsening of ME/CFS or FM symptoms.

To deliver the compositions of the present invention for the treatment of ME/CFS and/or FM, a hydrogen gas generator, hydrogen water generator, or hydrogen gas adding apparatus (e.g., a hydrogen gas supply (or gaseous hydrogen inhaler), hydrogen adding apparatus (or hydrogen water generator) as described above) that has been shown to have sufficient therapeutic efficacy and safety), non-destructive hydrogen-containing apparatus (a device that non-destructively dissolves hydrogen gas in a biological application solution such as intravenous solution sealed in a hydrogen-permeable bag), etc.) should be used.

The following examples will further illustrate the invention in detail, but the technical scope of the invention is not limited to these examples.

### EXAMPLE 1

Hydrogen gas inhalation improves/ameliorate myalgic encephalomyelitis/chronic fatigue syndrome (ME/CFS) and/or fibromyalgia (FM) symptoms and/or prevents worsening of symptoms.

### Case 1

### (1) Patient's Medical Condition

The patient is a Japanese male, 58 years old in 2021. In 2021, he was diagnosed as suffering from persistent general fatigue after exertion and impaired thinking and concentration for the past 8 years. There is a possibility that one of the causes is a head injury sustained while skiing 8 years ago. He was also examined for cerebrospinal fluid hypovolemia caused by a skiing head injury. Based on the diagnostic guidelines of the Japan Fatigue Society and the Canadian diagnostic criteria, the patient was diagnosed with myalgic encephalomyelitis/chronic fatigue syndrome. The patient's activity level was lower than that of a normal person, and the patient was advised to limit his activities to a level lower than that of a normal person's daily life because fatigue, pain, and decreased ability to think were extreme and recovery would be difficult.

The severity of the patient's symptoms based on the Canadian Standard Diagnosis were as follows:

(1) Fatigue—fatigue so marked that persistence and activity level are severely impaired: Severity 3 (severe)

(2) Fatigue after exertion—loss of physical and mental endurance, fatigue exacerbated by physical exertion: Severity 3 (severe)

(3) Prolonged recovery from exertion—recovery to pre-exertion activity level takes more than 24 hours: Severity 2 (Moderate)

(4) Sensitivity to light, noise, and odor: Severity 3 (severe)

(5) Pain—myalgia, arthralgia, headache: Severity 2 (moderate)

(6) Sleep disturbances—non-restorative sleep, insomnia, hypersomnia: Severity 2 (moderate)

(7) Language retrieval difficulties or speaking wrong words: Severity 2 (moderate)

(8) Confusion and poor concentration: Severity 2 (moderate)

(9) Memory impairment—Impairment of short-term memory: Severity 1 (mild)

(10) Shortness of breath with exertion: Severity 2 (moderate)

(11) Gastrointestinal disorders—diarrhea, irritable bowel syndrome: Severity 1 (mild)

(12) Repetitive sore throat: Severity 1 (mild)

(13) Dizziness or weakness on standing: Severity 1 (mild)

(14) Intolerance to heat/cold: Severity 1 (mild)

(15) Temperature changes, irregular body temperature, cold hands and feet: Severity 1 (mild)

(16) Repetitive influenza-like symptoms: Severity 0 (none)

(17) Facial flushing, sweating phenomenon: Severity 0 (none)

(18) Significant weight fluctuation: Severity 1 (mild)

(19) Lymph node tenderness, especially in the lateral aspect of the neck and axillae: Severity 0 (none)

(20) Muscle weakness: Severity 0 (none)

(21) New sensitivities to food/drugs/chemicals: Severity 1 (mild)

(2) Effect of Hydrogen Gas Inhalation after 2 Weeks of Inhalation

MiZ Corporation (Ofuna, Kamakura City) from Jun. 7, 2021, a hydrogen gas inhaler (Jobs-a, hydrogen concentration approx. 4%-5%, 100% hydrogen generation 200 ml/min) was used. Since the patient had a cold 3 days after the start of hydrogen inhalation, temporarily reduced the inhalation time to less than 3 hours. However, the patient inhaled for approximately 3 hours per day. The patient's head was foggy before inhalation of hydrogen, but after inhalation, the patient's head appeared to be clear and refreshed. This was the first time in several years that he had experienced such a feeling of brain fog clearing. The patient also felt a decrease in the frequency of headaches and general pain, and the number of analgesic medications she had to take decreased.

The patient's sympathetic nervous system was heightened before inhalation, but after inhaling hydrogen, the patient felt relaxed and drowsy.

(3) Effect of Hydrogen Gas Inhalation after 4 Weeks of Inhalation
(i) The symptoms that led to significant recovery based on the Canadian diagnostic criteria are itemized as follows:
   (1) Post-exertional fatigue—loss of physical and mental endurance, fatigue exacerbated by physical exertion: Pre-inhalation (Severity 3: severe)→Post-inhalation (Severity 2: moderate)
   (2) Prolonged recovery from exertion—recovery to pre-exertion activity level takes more than 24 hours: Pre-inhalation (Severity 2: moderate)→Post-inhalation (Severity 1: mild)
   (3)⋅ Fatigue—fatigue so marked that persistence and activity level are significantly impaired: Pre-inhalation (Severity 3: severe→Post-inhalation (Severity 1: mild or Severity 2: moderate)
   (4)⋅ Pain, headache: Pre-inhalation (Severity 2: moderate)→Post-inhalation (Severity 1: mild)
   (5)⋅ Decreased concentration: Pre-inhalation (Severity 2: moderate)→Post-inhalation (Severity 1: mild)
   (6)⋅ Difficulty in language retrieval or speaking wrong words: Pre-inhalation (Severity 2: Moderate)→Post-inhalation (Severity 1: Mild)
   (7)⋅ Repeated sore throat: Pre-inhalation (Severity 2: moderate)→Post-inhalation (Severity 1: mild)

No worsening of other symptoms was observed, suggesting that hydrogen suppressed the worsening of other symptoms.
(ii) Patient's Findings on Chronic Severe Fatigue

Inhalation of hydrogen gas greatly alleviated the chronic intense fatigue that causes bedridden conditions. Although the sudden onset of intense fatigue after exertion can still occur, the state of falling asleep one to two hours after exertion can be avoided by inhaling hydrogen for about 90 minutes.

Overall, the degree of fatigue varied from day to day, and although he still falls asleep two or three days a week, the patient's condition improved to the point where the patient felt as well as a healthy person more than half the days a week.

(iii) Patient's Perception of Physical Pain

Headaches occurred once or twice a week before hydrogen inhalation, but after hydrogen inhalation, the frequency of headaches decreased significantly and almost no headaches occurred. The frequency of analgesic medication was reduced to ¼ before hydrogen inhalation after hydrogen inhalation. Since last year, before the start of hydrogen gas inhalation, he has been doing muscle training and aerobic exercise for rehabilitation on good days to recover his physical strength and muscle power, but before hydrogen gas inhalation, muscle pain and joint pain after training were so severe that he had to take painkillers. After two weeks of hydrogen inhalation, the pain was greatly alleviated. Even when pain occurred due to training, the pain was almost completely eliminated the next day after 90 minutes of daily hydrogen inhalation. As for physical pain, the patient feels amazingly effective.

(iv) Patient's Impression about Brain Fog

The main complaint in the patient's medical condition is the decline in cognition, memory, and concentration, but the foggy feeling in the brain has been reduced by hydrogen gas inhalation. Before hydrogen gas inhalation, the patient had a hard time doing anything, and always had to endure and strain himself to do it, but now, after starting hydrogen inhalation, his head is clear. As for his cognitive ability, he feels that his ability to write sentences and summarize materials has returned to normal a little.

He sometimes experiences sudden onset of strong fatigue after working at a desk for one to two hours, and he has difficulty in speaking and comprehending sentences. Before the start of hydrogen inhalation, these symptoms did not improve easily. However, with the inhalation of hydrogen, such fatigue, as well as body aches, are now resolved in a shorter time (2 to 3 hours) than before hydrogen inhalation.

### (v) Summary of Symptoms.

Chronic fatigue, pain, and brain fog are clearly being relieved by hydrogen.

Post-exertional fatigue and sleep disturbances are still strong symptoms, but the degree of post-exertional fatigue has increased to a degree that resolves within 2 to 3 hours with hydrogen inhalation. These improvements are astounding considering that the patient had been suffering from the disorder for several years. Although sleep disturbances are still as strong as before, she has never been more sleepy after hydrogen inhalation.

It seems likely that the symptoms that can be immediately alleviated by hydrogen may have led to relatively early improvement and/or suppression of symptom worsening.

### Case 2

### Case 2: 2017 at the National Center for Neurology and Psychiatry (NCNP).

A 43-year-old woman diagnosed with ME/CFS had previously tried various treatments but her condition had not improved. Prior to hydrogen gas inhalation, she was unable to stand, stand, or maintain a seated posture due to significant impairment of upper and lower extremity function and trunk function, spending most of her day in bed and using a wheelchair for indoor mobility. She also exhibited symptoms of generalized pain, weakness, malaise, fatigue after exertion, sleep disturbance, low-grade fever, bradycardia, and hypotension.

She inhaled hydrogen gas for 6 hours per day starting at the end of August 2021 (Hydrogen gas inhaler: MiZ Corporation (Ofuna, Kamakura City), Jobs-α, hydrogen concentration Approx. 4%-5%, 100% hydrogen generation 200 ml/min). The results showed that sleep disturbance improved/ameliorated around 3 weeks after inhalation, and daily headache and migraine headache that occurred several times a day were alleviated. 9 weeks later, hydrogen gas inhalation still caused general pain, weakness, fatigue, tiredness after exertion, sleep disturbance, low fever, bradycardia, and low blood pressure. The symptom relief effect continued before hydrogen gas inhalation.

### Case 3

Case 3: An 18-year-old diagnosed with ME/CFS at the National Institute of Neurology and Psychiatry in April 2020.

The woman had tried various treatments but her medical condition had not improved. Prior to hydrogen gas inhalation, she had spent most of the day in bed with symptoms of low-grade fever, dizziness, hearing sensitivity, fatigue, decreased ability and desire to think, nausea, and headache.

She inhaled hydrogen gas for 5 hours per day starting at the end of August 2021 (Hydrogen gas inhaler: MiZ Corporation (Ofuna, Kamakura City), Jobs-a, hydrogen concentration Approx. 4%-5%, 100% hydrogen generation 200 ml/min).

As a result, she felt a little lighter and showed improvement in bowel movements and decreased motivation from about 3 weeks after inhalation. She also spent less time napping; at the end of eight weeks, she was still napping more days without naps and had more time for physical activity, although she still felt fatigued. She also indicated that she felt herself moving in the right direction. Subsequently, she observed symptom relief prior to hydrogen gas inhalation, including low fever, dizziness, hearing sensitivity, fatigue, decreased thinking and motivation, nausea, and headache.

### Case 4

Case 4: An 18-year-old diagnosed with both ME/CFS and FM at the National Center of Neurology and Psychiatry in August 2020.

The male patient had also tried various treatments to date but his condition had not improved. Prior to hydrogen gas inhalation, he had symptoms of fatigue, headache, muscle weakness, weakness, generalized pain, sleep disturbance, poor thinking, concentration, and memory, tinnitus, sensitivity to sound, and brain fog.

He inhaled hydrogen gas for 6 hours per day starting at the end of August 2021 (Hydrogen gas inhaler: MiZ Corporation (Ofuna, Kamakura City), Jobs-α, hydrogen concentration Approx. 4%-5%, 100% hydrogen generation 200 ml/min). After 9 weeks, the effects of hydrogen gas inhalation on headache and general pain continued, and the use of analgesics was reduced and “brain fog” was improved/ameliorated. Subsequently, he showed symptom relief before hydrogen gas inhalation, including fatigue, headache, muscle weakness, weakness, general body pain, sleep disturbance, impaired thinking, concentration, and memory, tinnitus, hypersensitivity to sound, and brain fog.

### INDUSTRIAL APPLICABILITY

The present invention enables ME/CFS and/or FM patients to improve/ameliorate and/or reduce the worsening of symptoms of ME/CFS and/or FM, such as fatigue, headache, body pain, poor thinking, poor concentration, and/or reduced activity levels associated with these symptoms, simply by administering hydrogen to them. Hydrogen itself has no known side effects and can enhance the patient's quality of life.

What is claimed is:

1. A method for ameliorating a symptom of myalgic encephalomyelitis/chronic fatigue syndrome (ME/CFS) and/or fibromyalgia (FM) and/or inhibiting worsening of a symptom in a patient with ME/CFS and/or FM, comprising:
administering to the patient a composition comprising hydrogen gas-containing gas, the hydrogen gas being the active ingredient.

2. The method of claim 1, wherein the symptom of ME/CFS includes at least one of fatigue, tiredness, shortness of breath with exertion, palpitations, muscle weakness, slight fever, muscle pain, joint pain, sore throat, headache, physical pain, generalized pain, weakness, cognitive dysfunction, memory impairment, difficulty with language retrieval, gastrointestinal disturbances, impaired concentration, flu-like symptoms, dizziness on standing, weakness, irregular body temperature, cold hands and feet, intolerance to heat and/or cold, perspiration, facial flushing, lymph node tenderness, significant weight fluctuations, tinnitus, confusion, sleep disturbances, dizziness when standing up, dizziness, hypersensitivity to light, noise, or odors, new acquired sensitivity to food, drugs, or chemicals, weakness, fatigue after exertion, bradycardia, decreased blood pressure, nausea, brain fog, and decreased activity level associated with any one of the above symptom(s).

3. The method of claim 1, wherein the symptom of FM includes at least one of slight fever of 38° C. or less, fatigue, tiredness weakness, stiffness of the hands and fingers, swollen fingers, joint pain, Raynaud's phenomenon, night sweats, irritable bowel syndrome, palpitations, dryness symptoms, dyspnea, dysphagia, interstitial cystitis-like symptoms, menstrual irregularities, dysmenorrhea, weight fluctuation, photosensitivity, intolerance to cold and heat, temporomandibular joint disorder, low blood pressure, various allergic symptoms, mitral valve deviation, itching, numbness in the extremities, hand tremors, dizziness, tinnitus, hearing loss, vision impairment, depressive symptoms, anxiety, agitation, sleep disturbances (hypersomnia, insomnia), decreased concentration, decreased attention span, amnesia, discomfort upon awakening, muscle weakness, decreased ability to think, concentrate, and remember, hypersensitivity to sound, brain fog, and decreased activity level associated with any one of the above symptom(s).

4. The method of claim 1, wherein the cause of ME/CFS is a viral infection.

5. The method of claim 1, wherein the hydrogen concentration of the hydrogen gas-containing gas is greater than zero (0) and less than 18.5 volume percent.

6. The method of claim 1, wherein the composition is administered to the patient by inhalation.

7. The method of claim 1, where the composition is made using a hydrogen gas generator.
